(19) United States
(12) Patent Application Publication
(10) Pub. No.: US 2026/0166301 A1
(43) Pub. Date: Jun. 18, 2026

(54) SYSTEMS AND METHODS FOR DETERMINING LOW PULSE PRESSURE EVENTS USING AN AUXILIARY MONITOR

(71) Applicant: Abiomed, Inc., Danvers, MA (US)

(72) Inventor: Christian Moyer, Danvers, MA (US)

(21) Appl. No.: 19/368,583

(22) Filed: Oct. 24, 2025

Related U.S. Application Data
(60) Provisional application No. 63/712,445, filed on Oct. 26, 2024.

(51) Int. Cl. A61M 60/585; A61M 60/13

(52) U.S. Cl. CPC A61M 60/585; A61M 60/13; A61M 2205/3344; A61M 2205/3561; A61M 2205/502; A61M 2205/583; A61M 2210/125; A61M 2230/04

(57) ABSTRACT

Methods and apparatus for an auxiliary monitor including a communication interface and at least one computer processor are described. The communication interface is configured to receive first data from a first patient monitoring device and second data from a second patient monitoring device, the first patient monitoring device including a heart pump, and the first data including an aortic pressure signal and a ventricular pressure signal measured during operation of the heart pump. The at least one computer processor is programmed to detect a timing of heartbeats in the aortic pressure signal and/or the ventricular pressure signal, determine a pulse pressure of the aortic pressure signal and/or the ventricular pressure signal within time windows associated with the timing of heartbeats, compare the determined pulse pressure against one or more metrics to identify one or more events, and output on a user interface, an indication of the one or more events.

### CROSS REFERENCE TO RELATED APPLICATIONS

This application claims the benefit under 35 U.S.C. § 119(e) to U.S. Provisional Ser. No. 63/712,445 , filed Oct. 26, 2024, and titled, “SYSTEMS AND METHODS FOR DISPLAY OF HEMODYNAMIC SIGNALS OR INDICATORS,” the contents of which is incorporated by reference herein in its entirety.

### FIELD OF THE INVENTION

This disclosure relates to using an auxiliary monitor coupled to multiple patient monitoring devices to determine one or more low pulse pressure events.

### BACKGROUND

Fluid pumps, such as blood pumps, are used in the medical field in a wide range of applications and purposes. An intravascular blood pump is a pump that can be advanced through a patient's vasculature, i.e., veins and/or arteries, to a position in the patient's heart or elsewhere within the patient's circulatory system. For example, an intravascular blood pump may be inserted via a catheter and positioned to span a heart valve. The intravascular blood pump is typically disposed at the end of the catheter. Once in position, the pump may be used to assist the heart and pump blood through the circulatory system and, therefore, temporarily reduce workload on the patient's heart, such as to enable the heart to recover after a heart attack. An exemplary intravascular blood pump is available from ABIOMED, Inc., Danvers, MA under the tradename Impella® heart pump.

Such pumps can be positioned, for example, in a cardiac chamber, such as the left ventricle, to assist the heart. In this case, the blood pump may be inserted via a femoral artery by means of a hollow catheter and introduced up to and into the left ventricle of a patient's heart. From this position, the blood pump inlet draws in blood and the blood pump outlet expels the blood into the aorta. In this manner, the heart's function may be replaced or at least assisted by operation of the pump.

An intravascular blood pump is typically connected to a respective external heart pump controller that controls the heart pump, such as motor speed, and collects and displays operational data about the blood pump, such as heart signal level, battery temperature, blood flow rate and plumbing integrity. An exemplary heart pump controller is available from ABIOMED, Inc. under the trade name Automated Impella Controller®. The controller may raise alarms when operational data values fall outside predetermined values or ranges, for example if a leak, suction, and/or pump malfunction is detected. The controller may include a video display screen upon which is displayed a graphical user interface configured to display the operational data and/or alarms.

### SUMMARY

In some embodiments an auxiliary monitor for a patient monitoring system is provided. The patient monitoring system includes a first patient monitoring device and a second patient monitoring device. The auxiliary monitor includes a communication interface and at least one computer processor. The communication interface is configured to receive first data from the first patient monitoring device and second data from the second patient monitoring device, wherein the first patient monitoring device and/or the second patient monitoring device includes a heart pump, and the first data and/or the second data includes an aortic pressure signal and a ventricular pressure signal measured during operation of the heart pump. The at least one computer processor is programmed to detect a timing of heartbeats in the aortic pressure signal and/or the ventricular pressure signal, determine a pulse pressure of the aortic pressure signal and/or the ventricular pressure signal within time windows associated with the timing of heartbeats, compare the determined pulse pressure against one or more metrics to identify one or more events, and output on a user interface, an indication of the one or more events.

In one aspect, the at least one computer processor is programmed to detect a timing of heartbeats by identifying heartbeats in the ventricular pressure signal. In another aspect, the at least one computer processor is programmed to determine a pulse pressure of the aortic pressure signal as a difference between a minimum value and a maximum value within each of the time windows. In another aspect, the one or more events includes one or more low pulse pressure events, and wherein the at least one computer processor is programmed to compare the determined pulse pressure against one or more metrics to identify one or more events by determining whether the pulse pressure for each time window is less than a threshold value, and identifying a low pulse pressure event when the pulse pressure for the time window is less than the threshold value. In another aspect, the threshold value is received via the user interface. In another aspect, the first patient monitoring device and/or the second patient monitoring device includes a heart pump, and the at least one computer processor is further programmed to generate a summarization of an interventional procedure for a patient associated with the first patient monitoring device and a second patient monitoring device, wherein generating a summarization of the interventional procedure comprises one or more of identifying one or more times during the interventional procedure when a low pulse pressure event was identified, determining a first average aortic pressure during the interventional procedure and a second average aortic pressure during time windows in which a low pulse pressure event was identified, or determining a first average flow through the heart pump during the interventional procedure and a second average flow through the heart pump during time windows in which a low pulse pressure event was identified, and outputting on a user interface, an indication of the one or more events comprises outputting an indication of the summarization of the interventional procedure.

In another aspect, the one or more events includes one or more low pulse pressure events, and wherein the at least one computer processor is programmed to compare the determined pulse pressure against one or more metrics to identify one or more events by detecting a low pulse pressure event when the pulse pressure for one or more time windows has decreased relative to baseline state. In another aspect, detecting a low pulse pressure event when the pulse pressure within a time window has decreased relative to baseline state includes determining that the pulse pressure has decreased from the baseline state by a first amount for a particular number of consecutive time windows.

In another aspect, outputting on a user interface, an indication of the one or more events comprises visually highlighting the one or more events on a plot displayed on the user interface. In another aspect, the at least one computer processor is further programmed to output a notification to a clinician about the one or more events. In another aspect, the at least one computer processor is further programmed to provide the indication of the one or more metrics as input to an algorithm used to assess weaning a patient associated with the first patient monitoring device and the second patient monitoring device from a heart pump.

In some embodiments, a method of detecting low pulse pressure events for a patient monitored with a first patient monitoring device and a second patient monitoring device is provided. The method includes receiving, by an auxiliary monitor communicatively coupled to the first patient monitoring device and the second patient monitoring device, first data from the first patient monitoring device and second data from the second patient monitoring device, wherein the first patient monitoring device and/or the second patient monitoring device includes a heart pump, and the first data and/or the second data includes an aortic pressure signal and a ventricular pressure signal measured during operation of the heart pump, detecting, by at least one computer processor, a timing of heartbeats in the aortic pressure signal and/or the ventricular pressure signal, determining a pulse pressure of the aortic pressure signal and/or the ventricular pressure signal within time windows associated with the timing of heartbeats, comparing the determined pulse pressure against one or more metrics to identify one or more events, and outputting on a user interface, an indication of the one or more events.

In one aspect, detecting a timing of heartbeats includes identifying heartbeats in the ventricular pressure signal. In another aspect, determining a pulse pressure of the aortic pressure signal comprises determining the pulse pressure as a difference between a minimum value and a maximum value of the aortic pressure signal within each of the time windows. In another aspect, the one or more events includes one or more low pulse pressure events, and comparing the determined pulse pressure against one or more metrics to identify one or more events includes determining whether the pulse pressure for each time window is less than a threshold value, and identifying a low pulse pressure event when the pulse pressure for the time window is less than the threshold value. In another aspect, the method further includes receiving the threshold value via the user interface. In another aspect, the first patient monitoring device and/or the second patient monitoring device includes a heart pump, and the method further includes generating a summarization of an interventional procedure for a patient associated with the first patient monitoring device and a second patient monitoring device, wherein generating a summarization of the interventional procedure comprises one or more of identifying one or more times during the interventional procedure when a low pulse pressure event was identified, determining a first average aortic pressure during the interventional procedure and a second average aortic pressure during time windows in which a low pulse pressure event was identified, or determining a first average flow through the heart pump during the interventional procedure and a second average flow through the heart pump during time windows in which a low pulse pressure event was identified, and outputting on a user interface, an indication of the one or more events comprises outputting an indication of the summarization of the interventional procedure.

In another aspect, the one or more events includes one or more low pulse pressure events, and comparing the determined pulse pressure against one or more metrics to identify one or more events includes detecting a low pulse pressure event when the pulse pressure for one or more time windows has decreased relative to baseline state. In another aspect, detecting a low pulse pressure event when the pulse pressure within a time window has decreased relative to baseline state includes determining that the pulse pressure has decreased from the baseline state by a first amount for a particular number of consecutive time windows.

In another aspect, outputting on a user interface, an indication of the one or more events comprises visually highlighting the one or more events on a plot displayed on the user interface. In another aspect, the method further includes outputting a notification to a clinician about the one or more events. In another aspect, the method further includes providing the indication of the one or more metrics as input to an algorithm used to assess weaning a patient associated with the first patient monitoring device and the second patient monitoring device from a heart pump.

### BRIEF DESCRIPTION OF DRAWINGS

FIG. 1 shows an illustrative cardiac support device that may be used to generate monitored data for use with some embodiments.

FIG. 2 shows an illustrative patient monitoring system that includes multiple patient monitoring devices and an auxiliary monitor, in accordance with some embodiments.

FIG. 3 shows a patient monitoring system that includes multiple patient monitoring devices, an auxiliary monitor, and an additional information source, in accordance with some embodiments.

FIG. 4 illustrates portions of a user interface of an auxiliary monitor, in accordance with one embodiment.

FIG. 5 illustrates portions of a user interface of an auxiliary monitor, in accordance with another embodiment.

FIG. 6 is a flowchart of a process for determining one or more patient-specific metrics using an auxiliary monitor, in accordance with some embodiments.

FIG. 7 is a flowchart of a process for identifying one or more acute events using an auxiliary monitor, in accordance with some embodiments.

### DETAILED DESCRIPTION

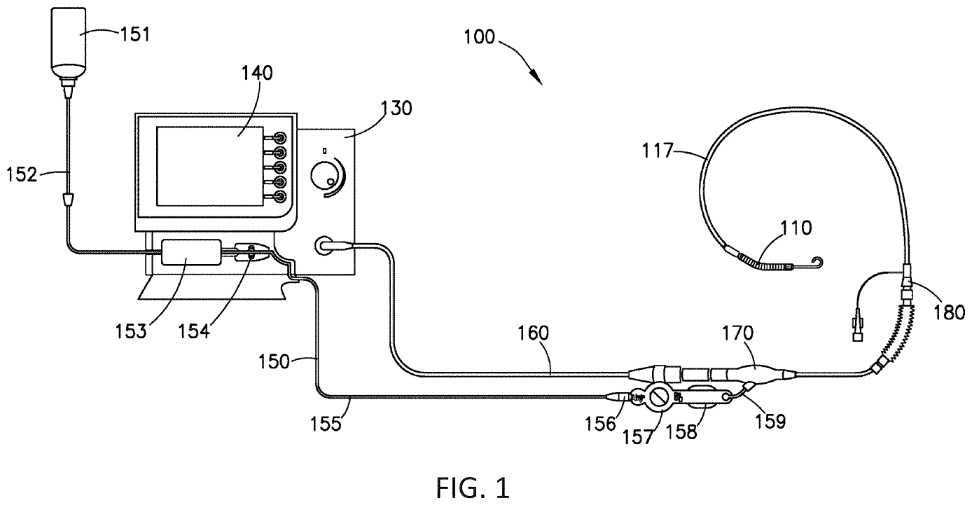

A circulatory support device (also referred to herein as a “heart pump” or simply a “pump”) may include a percutaneous, catheter-based device that provides hemodynamic support to the heart of a patient. As shown in FIG. 1, heart pump 110 may form part of a cardiac support system 100. Cardiac support system 100 also may include a controller 130 (e.g., an Automated Impella Controller®, referred to herein as an “AIC,” from ABIOMED, Inc., Danvers, Mass.), a display 140, a purge subsystem 150, a connector cable 160, a plug 170, and a repositioning unit 180. As shown, controller 130 may include display 140. Controller 130 may be configured to monitor and control operation of heart pump 110. During operation, purge subsystem 150 may be configured to deliver a purge fluid to heart pump 110 through catheter tube 117 to prevent blood from entering the motor (not shown) of the heart pump. In some implementations, the purge fluid is a dextrose solution (e.g., 5% dextrose in water with 25 or 50 IU/mL of heparin, although the solution need not include heparin in all embodiments). Connector cable 160 may provide an electrical connection between heart pump 110 and controller 130. Plug 170 may connect catheter tube 117, purge subsystem 150, and connector cable 160. In some implementations, plug 170 includes a storage device (e.g., a memory) configured to store, for example, operating parameters to facilitate transfer of the patient to another controller if needed. Repositioning unit 180 may be used to reposition heart pump 110 in the patient's heart.

As shown in FIG. 1, in some embodiments, the cardiac support system 100 may include a purge subsystem 150 having a container 151, a supply line 152, a purge cassette 153, a purge disc 154, purge tubing 155, a check valve 156, a pressure reservoir 157, an infusion filter 158, and a sidearm 159. Container 151 may, for example, be a bag or a bottle. As will be appreciated, in other embodiments the cardiac support system 100 may not include a purge subsystem. In some embodiments, a purge fluid may be stored in container 151. Supply line 152 may provide a fluidic connection between container 151 and purge cassette 153. Purge cassette 153 may control how the purge fluid in container 151 is delivered to heart pump 110. For example, purge cassette 153 may include one or more valves for controlling a pressure and/or flow rate of the purge fluid. Purge disc 154 may include one or more pressure and/or flow sensors for measuring a pressure and/or flow rate of the purge fluid. As shown, controller 130 may include purge cassette 153 and purge disc 154. Purge tubing 155 may provide a fluidic connection between purge disc 154 and check valve 156. Pressure reservoir 157 may provide additional filling volume during a purge fluid change. In some implementations, pressure reservoir 157 includes a flexible rubber diaphragm that provides the additional filling volume by means of an expansion chamber. Infusion filter 158 may help prevent bacterial contamination and air from entering catheter tube 117. Sidearm 159 may provide a fluidic connection between infusion filter 158 and plug 170. Although shown as having separate purge tubing and connector cable, it will be appreciated that in some embodiments, the cardiac support system 100 may include a single connector with both fluidic and electric lines connectable to the controller 130.

During operation, controller 130 may be configured to receive measurements from one or more pressure sensors (not shown) included as a portion of heart pump 110 and purge disc 154. Controller 130 may also be configured to control operation of the motor (not shown) of the heart pump 110 and purge cassette 153. As noted herein, controller 130 may be configured to control and measure a pressure and/or flow rate of a purge fluid via purge cassette 153 and purge disc 154. During operation, after exiting purge subsystem 150 through sidearm 159, the purge fluid may be channeled through purge lumens (not shown) within catheter tube 117 and plug 170. Sensor cables (not shown) within catheter tube 117, connector cable 160, and plug 170 may provide an electrical connection between components of the heart pump 110 (e.g., one or more pressure sensors) and controller 130. Motor cables (not shown) within catheter tube 117, connector cable 160, and plug 170 may provide an electrical connection between the motor of the heart pump 110 and controller 130. During operation, controller 130 may be configured to receive measurements from one or more pressure sensors of the heart pump 110 through the sensor cables (e.g., optical fibers) and to control the electrical power delivered to the motor of the heart pump 110 through the motor cables. By controlling the power delivered to the motor of the heart pump 110, controller 130 may be operable to control the speed of the motor.

Various modifications can be made to cardiac support system 100 and one or more of its components. For instance, one or more additional sensors may be added to heart pump 100. In another example, a signal generator may be added to heart pump 100 to generate a signal indicative of the rotational speed of the motor of the heart pump 110. As another example, one or more components of cardiac support system 100 may be separated. For instance, display 140 may be incorporated into another device in communication with controller 130 (e.g., wirelessly or through one or more electrical cables).

A heart pump (e.g., heart pump 110) may include a pressure sensor (e.g., an optical pressure sensor) configured to detect a pressure within the aorta of a patient's heart when the heart pump is properly positioned. The pressure signal sensed by the pressure sensor may be used, at least in part, to determine correct positioning of the heart pump within the patient's heart and/or to determine a blood flow rate through the heart pump when in operation. For instance, the pressure signal may be used in combination with a motor current signal received from a motor current sensor (not shown) and a set of stored values to determine a flow rate of blood through the heart pump. The differential pressure across the aortic valve may also indirectly be determined based on the pressure signal measuring the pressure in the aorta and the set of stored values.

Conventional medical devices include a controller and a display, which may be used to monitor one or more aspects of the operation of the medical device and/or the patient's physiology. The inventors have recognized and appreciated that when a patient is simultaneously using multiple medical devices (e.g., multiple patient monitoring devices), a healthcare worker (e.g., a physician) must monitor multiple controllers and displays to determine how the corresponding medical device is operating. Some embodiments of the technology described herein relate to an auxiliary monitor configured to receive data from multiple medical devices and display the data in a manner that provides a more intuitive and complete view of a patient's physiological condition when using the medical devices. For example, a physician may be able to monitor operation of multiple devices on a single display rather than having to monitor multiple displays connected to individual medical devices. In some embodiments, the auxiliary monitor includes at least one processor configured to process the data received from the multiple medical devices. In some instances, the data from multiple medical devices may be combined in a way that is not possible when the data from each medical device is considered in isolation.

The inventors have further recognized and appreciated that controllers of medical devices, such as cardiac support systems (e.g., controller 130 of heart pump 110), typically have limited functionality and processing power, which may be tailored to efficiently monitor and control operation of the devices. To this end, some embodiments of the technology described herein may extend the capabilities of such controllers by, for example, including an auxiliary monitor with additional processing capabilities and/or storage capacity for determining metrics (e.g., advanced metrics using one or more machine learning (ML) or artificial intelligence (AI) techniques) using data received from one or more controllers or other devices.

As described in further detail herein, the inventors have also recognized that some healthcare providers (e.g., surgeons or interventionalists) are typically unable to monitor some metrics displayed on the controller of a cardiac support system during a procedure due, at least in part, to the display having limited real estate, visibility, and/or access to the healthcare provider during the procedure. Some embodiments of the present disclosure enable the healthcare provider to view one or more metrics associated with operation of one or more medical devices without having to interact with or view the displays of the controllers associated with each individual device.

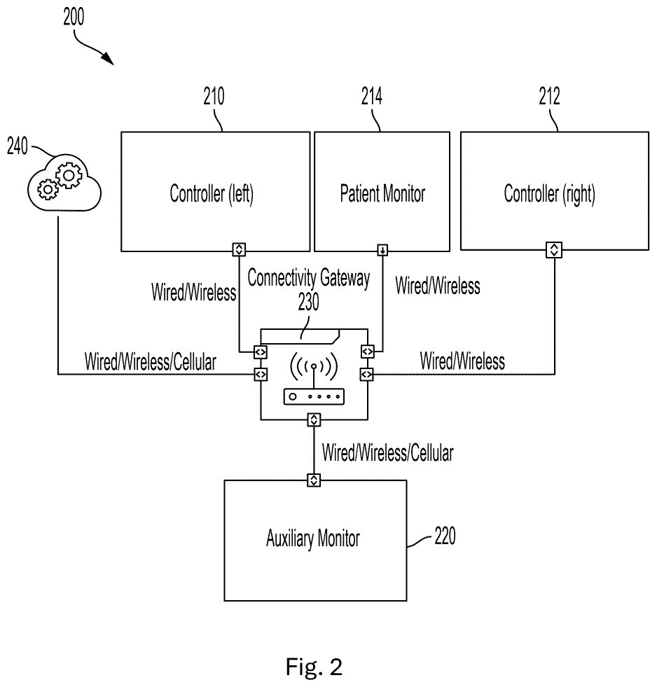

FIG. 2 illustrates a patient monitoring system 200 in accordance with some embodiments of the present disclosure. The patient monitoring system 200 includes a plurality of patient monitoring devices (e.g., AICs 210 and 212, and patient monitor 214) coupled to an auxiliary monitor 220 via a connectivity gateway 230. As will be appreciated, any suitable patient monitoring devices may be included in patient monitoring system 200. For instance, patient monitoring system may be implemented as a heart recovery system that includes one or more cardiac or cardio-pulmonary support consoles (e.g., an AIC, a blood oxygenation system, a balloon catheter and pump controller device) connected to connectivity gateway 230. Connectivity gateway 230 may provide a networking interface to transfer data between the patient monitoring devices and auxiliary monitor 220. As shown, the network connections between the patient monitoring devices and connectivity gateway may include wired (e.g., Ethernet, USB, coaxial cable) and/or wireless (e.g., WiFi, Cellular, Bluetooth) network interfaces. Data transmission may be achieved using any suitable transmission protocol examples of which include TCP and UDP. The auxiliary monitor 220 may be configured to encode/decode streams of TCP/UDP data and perform localized computation using the received data. The patient monitoring devices may include one or more controllers of a cardiac support system (e.g., AIC (Left) 210, AIC (Right) 212) configured to monitor parameters associated with a heart pump (e.g., pump speed, flow rate, purge flow rate, purge pressure), one or more patient monitoring devices (e.g., patient monitor 214) configured to monitor patient vital signs (e.g., heart rate, blood oxygenation, blood pressure, respiratory rate), and/or one or more other patient monitoring devices.

Data transmission between the patient monitoring devices and connectivity gateway 230 may be unidirectional and/or bidirectional. For instance, connectivity gateway 230 may be configured to receive monitored information from AICs 210 and 212 and patient monitor 214 and pass the monitored information (with or without transformation) to auxiliary monitor 220. In other embodiments, at least one of the connections between the patient monitoring devices and connectivity gateway 230 may be bidirectional. For instance, auxiliary monitor 220 may be configured to issue a request to connectivity gateway 230 for particular data from one of the patient monitoring devices, and in response, the corresponding patient monitoring device may provide the requested data to connectivity gateway 230, which in turn may provide the requested data to auxiliary monitor 220 for processing and/or display.

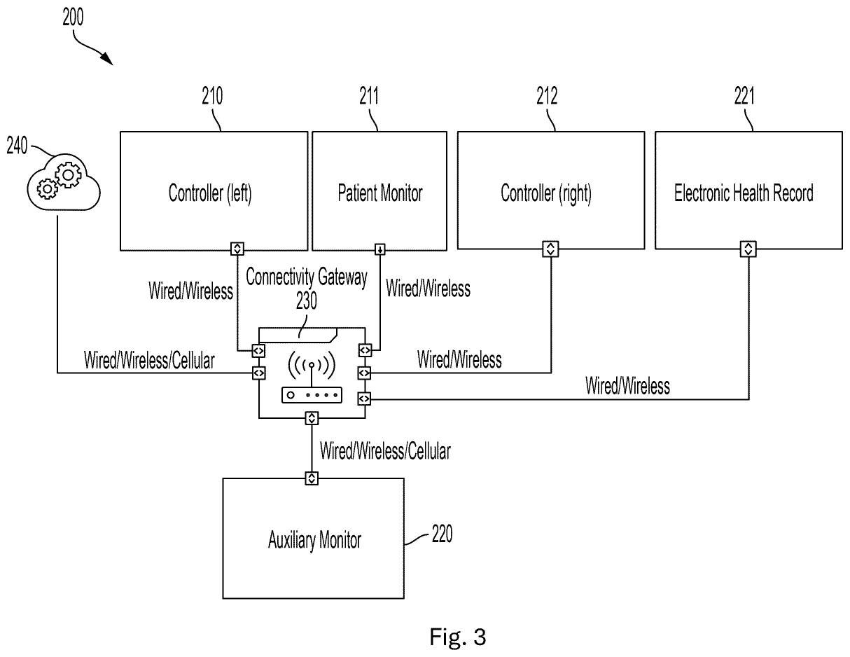

As described herein, auxiliary monitor 220 may be configured to determine one or more hemodynamic signals, predict one or more hemodynamic signals, and/or evaluate whether an alert threshold has been met. The auxiliary monitor 220 may display indications of data received via the connectivity gateway in a configurable user interface using one or more waveforms, values, trends and/or indicators. In still other embodiments, the auxiliary monitor may be configured to display patient data, such as data received from an electronic health record (EHR) stored in a hospital information system (see, e.g., FIG. 3).

In some embodiments, patient monitoring system 200 may be configured to segregate higher-risk control functions performed by the controller(s) for controlling operation of a connected device from lower-risk monitoring functions that collect and report data about the operation of the device and/or a physiological state of a patient associated with the device. By segregating control from monitoring, the auxiliary monitor 220 may be classified by the U.S. Food and Drug Administration (FDA) as a class II medical device (moderate risk) rather than a class III medical device (high risk). As such, software updates (e.g., new data processing algorithms, updates to data integration, etc.) for the auxiliary monitor 220 when classified as a class II medical device may not require as rigorous validation to ensure patient safety (e.g., through documentation provided to an approved by regulatory bodies) compared to devices classified as class III medical devices. The reduced requirements may allow for faster integration and/or distribution of new features in a clinical setting than if the updates were made directly to the controller(s). In some embodiments, updates to the auxiliary monitor 220 to include new features may be made remotely via connectivity gateway 230 as the features move through the verification and validation phase to further enhance rapid deployment of the new features.

As shown in FIG. 2, auxiliary monitor 220 may be configured to be coupled to multiple patient monitoring devices including, but not limited to, one or more AICs (e.g., AIC 210, 212), one or more patient monitors (e.g., patient monitor 214) or other patient monitoring devices.

In some embodiments, auxiliary monitor 220 may also be coupled to one or more additional information sources. For example, the auxiliary monitoring system may be connected to EHRs 221 stored by the hospital information system. In such embodiments, the auxiliary monitor 220 may be connected to the electronic EHRs via the connectivity gateway 230, such as via a wireless or wired connection. In some embodiments, the EHR may be configured to transmit patient data to the auxiliary monitor (e.g., via the gateway). In other embodiments, pump data may be transferred from one of the devices to the auxiliary monitor and thereafter to the EHR (e.g., for storage in a patient record). As will be appreciated and as described herein, appropriate security protocols may be in place to safeguard patient data being received and/or transferred to the EHR from the auxiliary monitor.

Each of the coupled patient monitoring devices and/or additional information sources may be associated with its own information stream that includes data corresponding to one or more monitored parameters associated with the corresponding device. In some embodiments, auxiliary monitor 220 may be configured to leverage multiple information streams received from the multiple devices and/or information sources to perform data processing that is not possible when only a single information stream is considered. In this way, the information provided and/or displayed by auxiliary monitor 220 may not be merely duplicative of that shown on the individual patient monitoring devices (e.g., AICs 210, 212, an ECMO device, etc.) or in an additional information source (e.g., the EHRs). Instead, information provided and/or displayed by the auxiliary monitor can include information determined, at least in part, on information from all or a subset of the coupled patient monitoring devices and/or additional information sources. In this regard, one or more of the algorithms disclosed herein may include data received from one or more of the patient monitoring devices and from one or more of the additional information sources (e.g., the EHR).

As described in more detail herein, a patient may have multiple cardiac support devices (e.g., a right system and a left system). Metrics that rely on information from both of the cardiac support devices may be determined and recommendations on how to modify operation of one or both of the cardiac support devices to optimize patient outcomes may be provided to guide treatment. For instance, a recommendation to increase or decrease support for one or both of the cardiac support devices may be determined. Algorithms for escalation or weaning may use one or more models that predict, based on current information from multiple information streams, how a change in the level of support may affect the cardiac and/or overall health of the patient (e.g., as the patient is working toward a particular goal, such as weaning off of a device). In this way, the auxiliary monitor 220 may be configured to provide a physician or other healthcare provider a more integrated or complete view of a patient's health than can be obtained from viewing multiple displays associated with individual patient monitoring devices.

In some embodiments, the auxiliary monitor 220 may be implemented as a desktop portable device or pole mountable unit. In some embodiments, the auxiliary monitor 220 may include a touch screen display that enables a healthcare professional to interact with the display to reconfigure what is displayed on the auxiliary monitor 220 as desired.

In some embodiments, the auxiliary monitor 220 may be configured to send one or more computed values via connectivity gateway 230 to a web-based service, such as cloud service 240. Cloud service 240 may be configured to re-display the computed values, or information derived based on the computed values, via a web-based interface (e.g., a web browser).

In some embodiments, auxiliary monitor 220 may be configured for use as a sensor gateway. The sensor gateway may be configured to combine parameters for sensor fusion and/or to provide (e.g., stream) sensor fusion data to a web-based services (e.g., cloud service 240). For instance, in some embodiments auxiliary monitor 220 may send data to another computing device (not shown) configured as a sensor gateway. The sensor gateway may, in turn, provide sensor fusion data to one or more other devices via, for example, connectivity gateway 230.

In some embodiments, auxiliary monitor 220 is configured to be coupled to one or more additional devices. Such additional devices may provide information that relates to and/or may be informed by context of hemodynamics support devices (e.g., one or more heart pumps). Non-limiting examples of such additional devices include infusion pumps, ventilators, ECMO machines, dialysis machines, devices for point-of-care blood measurements, imaging devices for point-of-care imaging. In some embodiments, the additional devices coupled to auxiliary monitor 220 may provide high-fidelity, continuous signals from one or more physiological measurements or therapies. The continuous signals from such devices may provide context to the measurements and/or algorithms used by a hemodynamic support device, such as a heart pump. Additionally, data from a hemodynamics support device may provide context to measurements and/or therapies of one or more of the additional devices coupled to the auxiliary monitor 220.

Some exemplary advantages of a patient monitoring system configured in accordance with the technology described herein include, but are not limited to, enabling advanced metrics or indicators from patient monitoring devices to be displayed in a surgical and interventional setting, enabling surgeons or interventionalists to view cardiac catheter data and advanced metrics on a secondary screen when the primary screen is typically inaccessible to them. Such a system may provide a platform for integrating metrics from other instruments or devices into a single display, provide a platform that can maintain a case context across multiple devices (e.g., maintain a history following a pump swap for escalation of support), and/or provide a platform to consolidate data for single or multiple patients.

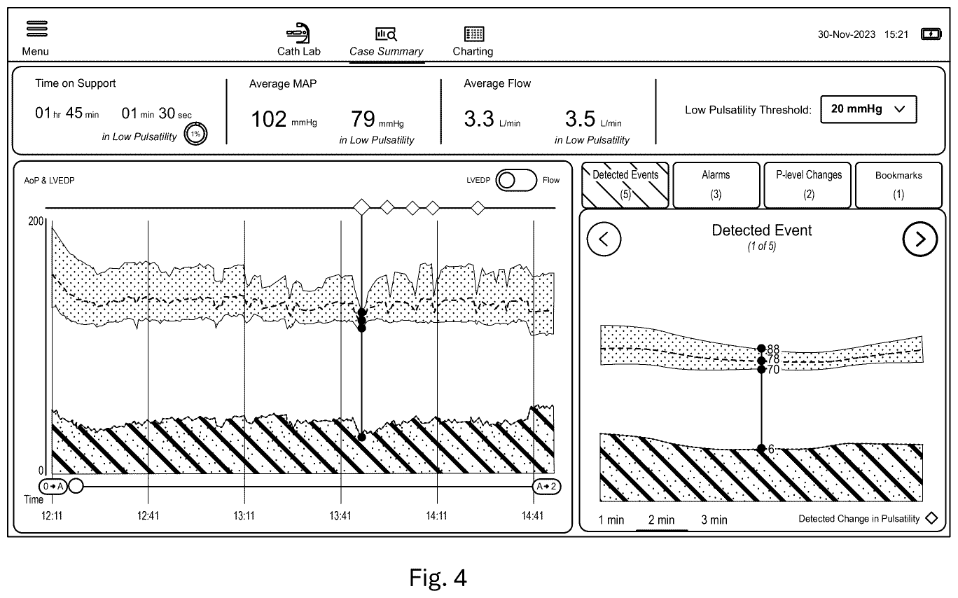

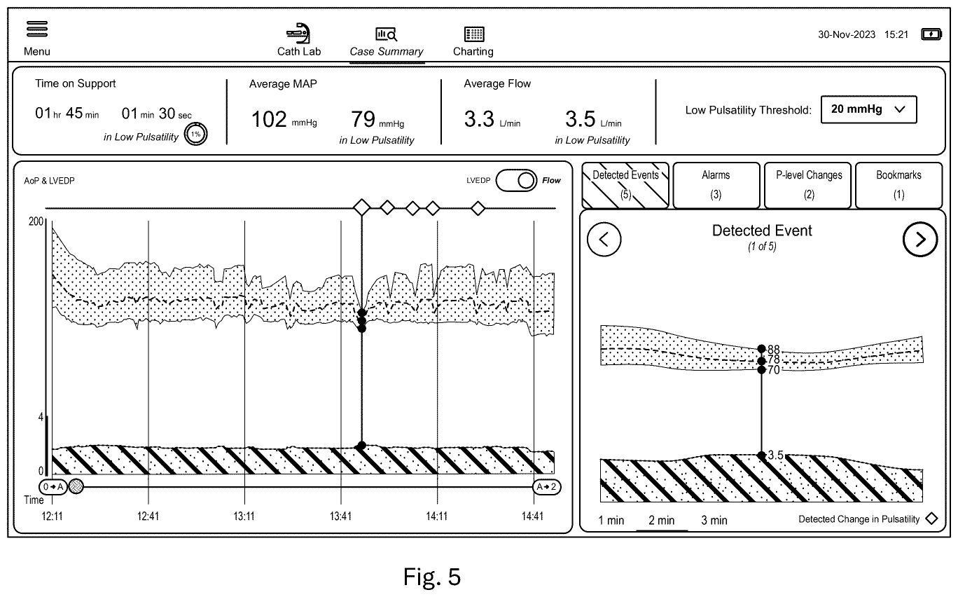

The inventors have recognized that advantages may be realized by summarizing data from an interventional procedure, such as a high-risk protected coronary intervention (HRPCI). For example, in some embodiments, the controller and/or auxiliary monitor may employ one or more algorithms useful to highlight areas of interest for a user and/or to explain the benefits of support (e.g., mechanical circulatory support) during the riskier periods of the case. FIGS. 4 and 5 illustrate examples of a user interface configured to employ one or more of these algorithms.

In some embodiments, a first algorithm may include a detection and averaging tool for periods of low pulse pressure during the case, which may be referred to as LowPP. In some embodiments, this LowPP algorithm starts with a basic threshold, set by the user, on what they define as a low pulse pressure for their patient. In some embodiments, this threshold pressure may be between 10 mmHg and 40 mmHg, and may depend on the patient and user preference. In some embodiments, the algorithm may then analyze an entire case worth of aortic and ventricular pressure signals. In some embodiments, heart beats may be detected using a heartbeat detection algorithm, which may identify a rise in pressure concordant with aventricular contraction. In some embodiments, once a beat is detected, the “pulse pressure” or “pulsatility” can be computed as maximum - minimum pressure within a beat. This may form the basis to test against the LowPP threshold. In some embodiments, ventricular pressure may be used to detect beats confidently, even when the aortic pressure is low / non pulsatile. In some embodiments, once the pulse pressure is computed, and a threshold is set, the algorithm may identify all of the periods where the pulse pressure was less than the threshold. This may enable the summarization, which may include: a) time of the case (absolute and % basis) with LowPP, b) average aortic pressure (MAP) during entire case and during periods of LowPP, c) average flow of a MCS support device during entire case and during periods of LowPP. In some embodiments, such a summarization may allow a physician to appreciate how complex a case was (i.e., how often was patient in LowPP state), and demonstrate the need and/or benefits of mechanical circulatory support, such as by showing the supported pressure and the flow provided during those same periods.

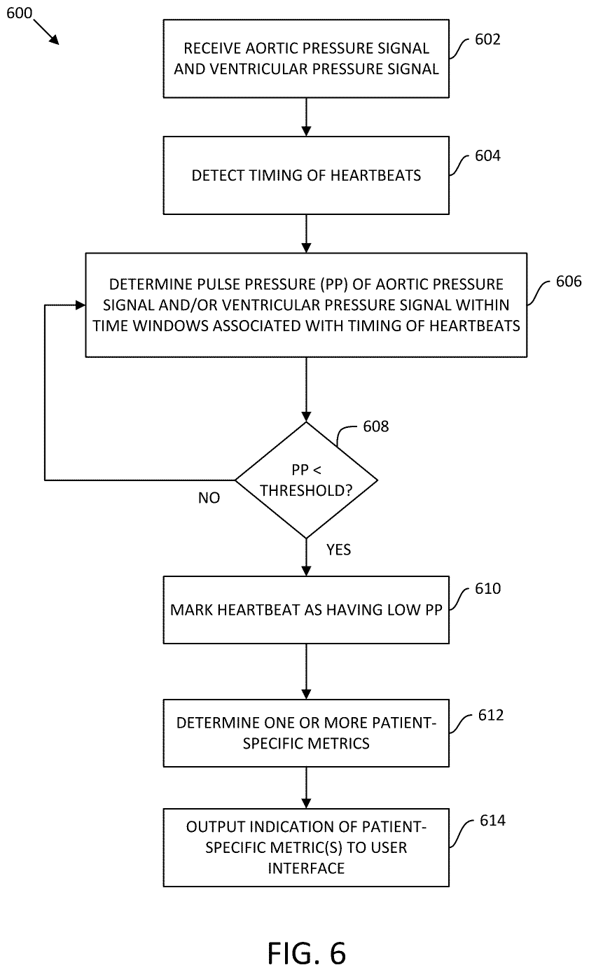

FIG. 6 is a flowchart of a process 600 for determining one or more patient-specific metrics using an auxiliary monitor communicatively coupled to a first patient monitoring device and a second patient monitoring device, in accordance with some embodiments. Process 600 may begin in act 602, where an aortic pressure signal and a ventricular pressure signal measured by one or more pressure sensors associated with a heart pump arranged in the left heart of a patient are received. It should be appreciated that process 600 may be performed as the aortic pressure signal and the ventricular pressure sensor are being sensed (or derived based on other signals) and/or process 600 may be performed based on stored aortic and ventricular pressure signals sensed, for example, during a procedure, such an interventional procedure (e.g., a high-risk protected coronary intervention).

Process 600 may then proceed to act 604, where a set of heartbeats are detected in the aortic pressure signal and/or the ventricular pressure signal. In some embodiments, the set of heartbeats may be detected using the ventricular pressure signal, for example, when the aortic pressure signal is low and/or non-pulsatile. Process 600 may then proceed to act 606, where a pulse pressure (also referred to herein as “pulsatility” of a pressure signal) may be determined for the aortic pressure signal and/or the ventricular pressure signal. For instance, the timing of detected heartbeats may be used to define a set of time windows within each of which the pulse pressure (PP) is determined. Process 600 may then proceed to act 608, where it is determined whether the pulse pressure for a particular time window is less than a threshold value. In some embodiments, the threshold value may set by a user in response to user input provided via a user interface (e.g., a user interface associated with the auxiliary monitor). If it is determined in act 608 that the pulse pressure is not less than the threshold, process 600 returns to act 606, were the pulse pressure is determined for additional time windows associated with the timing of the heartbeats in the set of heartbeats. When it is determined in act 608 that the pulse pressure for a time window is less than the threshold value, process 600 may proceed to act 610, where the heartbeat and/or the time window associated with the heartbeat is marked as having a low pulse pressure event.

After low pulse pressure event(s) have been determined for the time windows associated with the heartbeats, process 600 may proceed to act 612, where one or more patient-specific metrics may be determined based on the identified low pulse pressure events. Non-limiting examples of patient-specific metrics are described herein. For example, patient-specific metrics may include, but are not limited to, metrics associated with an interventional procedure such as a HRPCI procedure. In some embodiments, patient-specific metrics may include one or more of: a time of the procedure (e.g., an absolute time and/or percentage of the time of the procedure) associated with low pulse pressure events, a mean aortic pressure (MAP) during the entire procedure and/or during time windows of the procedure associated with low pulse pressure events, or an average flow of blood through a heart pump during the entire procedure and/or during time windows of the procedure associated with low pulse pressure events. Process 600 may then proceed to act 614, where an indication of the patient specific metric(s) is output to a user interface (e.g., a user interface associated with the auxiliary monitor). The indication of the patient specific metric(s) may be output as one or more numerical values, one or more graphs, a highlighted portion of one or more graphs or other user interface elements, or in any other suitable way.

In some embodiments, a second algorithm may be configured to detect specific, acute events of a drop in pulse pressure. In such embodiments, this detection algorithm may also include a version of a LowPP detection. In such embodiments, this algorithm may focus on drops in pulse pressure (not necessarily an absolute LowPP Threshold). In such embodiments, the algorithm may be configured to what a pressure drop means, how acute it is, how long it lasts, as examples. In some embodiments, the algorithm may also start with detecting beats and computing pulse pressure from beats as described above. However, in some embodiments, this second algorithm may include one or more additional filters to define an “event” and “acute”. In some embodiments, this definition may be derived by using a baseline state, using the systolic and mean pressure as checks, and defining time and magnitude bounds on what a meaningful drop in PP is and how long it would last. It also may have some hysteresis built in, such that you can't keep detected events consecutively. In some embodiments, this tool output may include a time marker of an event, which can then be used on the User Interface (e.g., on a Case Summary view) to visually highlight that event, what the drop looked like, and what the pressures and flows were during this event. The tool output also may be used to see how many events there where, how long they lasted, and how quickly the patient recovered from them. In some embodiments, this may include an entirely graphical/visual summary on the user interface. As will be appreciated, the output also could include a notification to the clinician. In still other embodiments, the algorithm may be configured such that the events may be “parametrizing”, allowing for computation of how long they last, how quickly a patient recovers, and if those patterns change over successive events in a case. In some embodiments, these outputs could be used as predictive indicators of patient outcomes, and in some embodiments, may be useful as inputs for other algorithms that could be incorporated into the user interface (e.g., an algorithm to assess weaning of mechanical circulatory support for the patient).

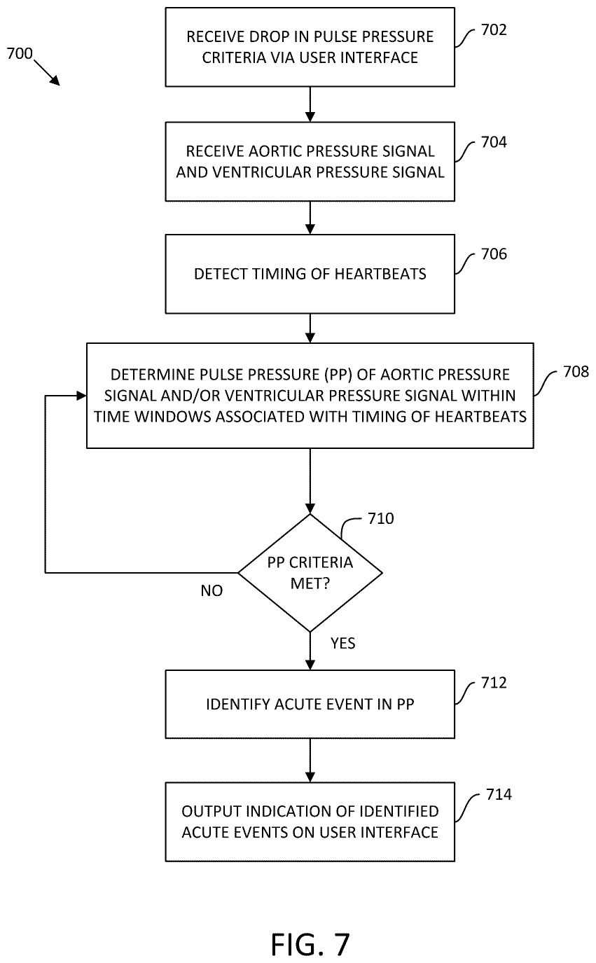

FIG. 7 is a flowchart of a process 700 for determining one or more acute events of reduced pulse pressure using an auxiliary monitor communicatively coupled to a first patient monitoring device and a second patient monitoring device, in accordance with some embodiments. Process 700 may begin in act 702, where criteria for detecting an acute event of reduced pulse pressure is received via a user interface. For instance, rather than using a single set threshold for comparing pulse pressure within individual time windows, as described in connecting with process 600 of FIG. 6, process 700 may evaluate pulse pressure across one or more time windows using other criteria including, but not limited to, an intensity of the pulse pressure relative to a baseline status of the patient, and a time of the reduced pulse pressure. Acts 704-708 of process 700 may proceed in substantially the same way as acts 602-606 of process 600, and are not repeated for brevity.

Process 700 may then proceed to act 710, where it is determined whether the pulse pressure(s) determined for one or more time windows meet the criteria for identifying an acute event of reduced pulse pressure received in act 702. If it is determined in act 710 that the pulse pressure(s) do not meet the criteria, process 700 returns to act 708, were additional pulse pressures are determined. When it is determined in act 710 that the pulse pressure for a time window meets the acute event criteria, process 700 may proceed to act 712, where an acute event of reduced pulse pressure is identified.

After acute event(s) of reduced pulse pressure have been determined for the time windows associated with the heartbeats, process 700 may proceed to act 714, where an indication of the identified acute event(s) may be output to a user interface (e.g., a user interface associated with the auxiliary monitor). The indication of the acute events may be output as one or more numerical values, one or more graphs, a highlighted portion of one or more graphs or other user interface elements, or in any other suitable way.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above, and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. An auxiliary monitor for a patient monitoring system, the patient monitoring system including a first patient monitoring device and a second patient monitoring device, the auxiliary monitor comprising:
a communication interface configured to receive first data from the first patient monitoring device and second data from the second patient monitoring device, wherein the first patient monitoring device and/or the second patient monitoring device includes a heart pump, and the first data and/or the second data includes an aortic pressure signal and a ventricular pressure signal measured during operation of the heart pump; and
at least one computer processor programmed to:
detect a timing of heartbeats in the aortic pressure signal and/or the ventricular pressure signal;
determine a pulse pressure of the aortic pressure signal and/or the ventricular pressure signal within time windows associated with the timing of heartbeats;
compare the determined pulse pressure against one or more metrics to identify one or more events; and
output on a user interface, an indication of the one or more events.

2. The auxiliary monitor of claim 1, wherein the at least one computer processor is programmed to detect a timing of heartbeats by identifying heartbeats in the ventricular pressure signal.

3. The auxiliary monitor of claim 1, wherein the at least one computer processor is programmed to determine a pulse pressure of the aortic pressure signal as a difference between a minimum value and a maximum value within each of the time windows.

4. The auxiliary monitor of claim 1, wherein the one or more events includes one or more low pulse pressure events, and wherein the at least one computer processor is programmed to compare the determined pulse pressure against one or more metrics to identify one or more events by:
determining whether the pulse pressure for each time window is less than a threshold value; and
identifying a low pulse pressure event when the pulse pressure for the time window is less than the threshold value.

5. The auxiliary monitor of claim 4, wherein the threshold value is received via the user interface.

6. The auxiliary monitor of claim 4, wherein the first patient monitoring device and/or the second patient monitoring device includes a heart pump, the at least one computer processor is further programmed to:
generate a summarization of an interventional procedure for a patient associated with the first patient monitoring device and a second patient monitoring device, wherein generating a summarization of the interventional procedure comprises one or more of:
identifying one or more times during the interventional procedure when a low pulse pressure event was identified;
determining a first average aortic pressure during the interventional procedure and a second average aortic pressure during time windows in which a low pulse pressure event was identified; or
determining a first average flow through the heart pump during the interventional procedure and a second average flow through the heart pump during time windows in which a low pulse pressure event was identified, and
wherein outputting on a user interface, an indication of the one or more events comprises outputting an indication of the summarization of the interventional procedure.

7. The auxiliary monitor of claim 1, wherein the one or more events includes one or more low pulse pressure events, and wherein the at least one computer processor is programmed to compare the determined pulse pressure against one or more metrics to identify one or more events by:
detecting a low pulse pressure event when the pulse pressure for one or more time windows has decreased relative to baseline state.

8. The auxiliary monitor of claim 7, wherein detecting a low pulse pressure event when the pulse pressure within a time window has decreased relative to baseline state comprises:
determining that the pulse pressure has decreased from the baseline state by a first amount for a particular number of consecutive time windows.

9. The auxiliary monitor of any of claims 1-8, wherein outputting on a user interface, an indication of the one or more events comprises visually highlighting the one or more events on a plot displayed on the user interface.

10. The auxiliary monitor of any of claims 1-8, wherein the at least one computer processor is further programmed to output a notification to a clinician about the one or more events.

11. The auxiliary monitor of any of claims 1-8, wherein the at least one computer processor is further programmed to provide the indication of the one or more metrics as input to an algorithm used to assess weaning a patient associated with the first patient monitoring device and the second patient monitoring device from a heart pump.

12. A method of detecting low pulse pressure events for a patient monitored with a first patient monitoring device and a second patient monitoring device, the method comprising:
receiving, by an auxiliary monitor communicatively coupled to the first patient monitoring device and the second patient monitoring device, first data from the first patient monitoring device and second data from the second patient monitoring device, wherein the first patient monitoring device and/or the second patient monitoring device includes a heart pump, and the first data and/or the second data includes an aortic pressure signal and a ventricular pressure signal measured during operation of the heart pump;
detecting, by at least one computer processor, a timing of heartbeats in the aortic pressure signal and/or the ventricular pressure signal;
determining a pulse pressure of the aortic pressure signal and/or the ventricular pressure signal within time windows associated with the timing of heartbeats;
comparing the determined pulse pressure against one or more metrics to identify one or more events; and
outputting on a user interface, an indication of the one or more events.

13. The method of claim 12, wherein detecting a timing of heartbeats comprises identifying heartbeats in the ventricular pressure signal.

14. The method of claim 12, wherein determining a pulse pressure of the aortic pressure signal comprises determining the pulse pressure as a difference between a minimum value and a maximum value of the aortic pressure signal within each of the time windows.

15. The method of claim 12, wherein the one or more events includes one or more low pulse pressure events, and comparing the determined pulse pressure against one or more metrics to identify one or more events comprises:
determining whether the pulse pressure for each time window is less than a threshold value; and
identifying a low pulse pressure event when the pulse pressure for the time window is less than the threshold value.

16. The method of claim 15, further comprising receiving the threshold value via the user interface.

17. The method of claim 15, wherein the first patient monitoring device and/or the second patient monitoring device includes a heart pump, and the method further comprises:
generating a summarization of an interventional procedure for a patient associated with the first patient monitoring device and a second patient monitoring device, wherein generating a summarization of the interventional procedure comprises one or more of:
identifying one or more times during the interventional procedure when a low pulse pressure event was identified;
determining a first average aortic pressure during the interventional procedure and a second average aortic pressure during time windows in which a low pulse pressure event was identified; or
determining a first average flow through the heart pump during the interventional procedure and a second average flow through the heart pump during time windows in which a low pulse pressure event was identified, and
wherein outputting on a user interface, an indication of the one or more events comprises outputting an indication of the summarization of the interventional procedure.

18. The method of claim 12, wherein the one or more events includes one or more low pulse pressure events, and comparing the determined pulse pressure against one or more metrics to identify one or more events comprises:
detecting a low pulse pressure event when the pulse pressure for one or more time windows has decreased relative to baseline state.

19. The method of claim 18, wherein detecting a low pulse pressure event when the pulse pressure within a time window has decreased relative to baseline state comprises:
determining that the pulse pressure has decreased from the baseline state by a first amount for a particular number of consecutive time windows.

20. The method of any of claims 12-19, wherein outputting on a user interface, an indication of the one or more events comprises visually highlighting the one or more events on a plot displayed on the user interface.

21. The method of any of claims 12-19, further comprising outputting a notification to a clinician about the one or more events.

22. The method of any of claims 12-19, further comprising providing the indication of the one or more metrics as input to an algorithm used to assess weaning a patient associated with the first patient monitoring device and the second patient monitoring device from a heart pump.
